(19) United States
(12) Patent Application Publication
(10) Pub. No.: US 2026/0174651 A1
(43) Pub. Date: Jun. 25, 2026

(54) Pearlescent Wash Composition

(71) Applicant: Conopco, Inc., d/b/a UNILEVER, Englewood Cliffs, NJ (US)

(72) Inventors: Haley Ruth SHELLEY, New Haven, CT (US); Douglas John HIBAN, Newtown, CT (US)

(73) Assignee: Conopco, Inc., d/b/a UNILEVER, Englewood Cliffs, NJ (US)

(21) Appl. No.: 18/987,867

(22) Filed: Dec. 19, 2024

(51) Int. Cl. A61K 8/37; A61Q 5/02; A61Q 19/10

(52) U.S. Cl. CPC A61K 8/37; A61Q 5/02; A61Q 19/10; A61K 2800/30; A61K 2800/42

(57) ABSTRACT

The present invention is directed to an isotropic and pearlescent wash composition. The composition is stable, comprises pearlescent agent and peroxisome proliferator-activated receptor (PPAR) agonist. The pearlescent agent is homogeneously dispersed when the wash composition is substantially free of polymeric thickening agent and suspending agent.

### FIELD OF THE INVENTION

The present invention is directed to a pearlescent wash composition. More particularly, the composition is directed to a pearlescent and isotropic wash composition having amphoteric and anionic surfactant, and surprisingly, a homogeneously dispersed pearlescent agent even when the wash composition is substantially free of a polymeric thickener and suspending agent. The composition comprises a peroxisome proliferator-activated receptor (PPAR) agonist, and surprisingly, is stable, maintains superior color and fragrance characteristics, and can be formulated substantially free of at least one of a sulfate-based surfactant, paraben, hydantoin, isothiazolinone, phthalate, soap, acrylate-based thickener, dioxane and silicone oil.

### BACKGROUND OF THE INVENTION

Aqueous wash compositions, such as detergents, face washes, shampoos and body washes, are common and enjoyed by many consumers. Such compositions typically have water as the predominant ingredient, and they are often sold in plastic bottles, sachets or tubes. The compositions are conventionally formulated to have a viscosity that is customary for consumer use and easy for evacuation from the package they are sold in.

While consumers want products with traditional wash composition characteristics, many are demanding compositions that deliver lasting fragrance and are mild to their skin and hair. Notwithstanding, wash compositions with mild surfactants are known to present challenges when suspending ingredients and stabilizing viscosity. Often, the solution to such challenges is solved with the use of polymeric thickener or suspending agent or both. While such thickeners can, for example, aid in viscosity stabilization, they can be unreliable when product is subject to temperature fluctuations and certain consumers do feel polymeric thickeners and suspending agents impede product foam richness they often desire.

It is of increasing interest to develop a wash composition that is mild, imparts a lasting fragrance, has a rich foam profile, can maintain viscosity and suspend product components without needing a polymeric thickener and suspending agent. The present invention, therefore, is directed to a pearlescent and isotropic wash composition having amphoteric and anionic surfactant, and surprisingly, a homogeneously dispersed pearlescent agent even when the wash composition is substantially free of a polymeric thickener and suspending agent. The composition comprises a peroxisome proliferator-activated receptor (PPAR) agonist, is stable, maintains superior color and fragrance characteristics, and can be formulated substantially free of at least one of a sulfate-based surfactant, paraben, hydantoin, isothiazolinone, phthalate, soap, acrylate-based thickener, dioxane and silicone oil.

### Additional Information

Efforts have been disclosed for making wash compositions. In World Application WO17035766A1, hair cleansing compositions having surfactants, polymeric thickeners and lotus flower oil are described.

Other efforts have been disclosed for making wash compositions. In U.S. Patent Application Publication No. 2023/0414468 A1, a wash composition that does not irritate the eyes and that has anionic surfactant, amphoteric or zwitterionic surfactant or both, and nonionic surfactant or cationic polymer or a mixture thereof is described.

Further efforts have been disclosed for making wash compositions. In U.S. Patent Application Publication No. 2023/045404 A1, an isotropic concentrate that is hydratable and suitable to transform into an end use wash composition upon hydration is described.

Even other efforts have been disclosed for making wash compositions. In U.S. Patent Application Publication No. 2016/0319222 A1, a composition with surfactant and potassium chloride to impart pearlescence is described.

None of the additional information describes a wash composition that comprises a homogeneously dispersed pearlescent agent as claimed herein.

### SUMMARY OF THE INVENTION

In a first aspect the present invention is directed to a wash composition comprising:
a. from 1.5 to 4.5%, and preferably, from 1.8 to 3.8%, and most preferably, from 2 to 3.3% or from 2 to 3% or from 2.2 to 2.9% or from 2.3 to 2.8% by weight of an anionic surfactant comprising at least 80% by weight of acyl isethionate and acyl taurate based on total weight of the anionic surfactant;
b. from 3 to 10%, and preferably, from 4 to 8%, and most preferably, from 5 to 7.5% or from 5.5 to 7.5% or from 6 to 7.5% or from 6.5 to 7.25% by weight amphoteric surfactant;
c. from 0.08 to 1%, and preferably, from 0.1 to 0.8%, and most preferably, from 0.12 to 0.7% or from 0.12 to 0.6% or from 0.15 to 0.5% or from 0.15 to 0.35% or from 0.15 to 0.25% by weight of a peroxisome proliferator-activated receptor (PPAR) agonist, the PPAR agonist comprising at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist;
d. from 0.05 to 1%, and preferably, from 0.07 to 0.5%. and most preferably, from 0.08 to 0.4% or from 0.08 to 0.25%, or from 0.08 to 0.2% or from 0.09 to 0.16%, or from 0.09 to 0.15 or from 0.1 to 0.14% by weight of a pearlescent agent, the pearlescent agent comprising from 0 to 100%, and preferably, from 15 to 100%, and most preferably, from 40 to 100% or from 50 to 100%, or from 60 to 100% or from 75 to 100% or from 75 to 90% or from 80 to 95% or from 90 to 99% or from 92 to 100% or from 96 to 100% or 100% by weight glycol distearate; and
e. from 70 to 92%, and preferably, from 72 to 88%, and most preferably, from 75 to 86% or from 78 to 85% by weight water,
   wherein:
i) the composition is substantially free of polymeric thickener and suspending agent;
ii) the pearlescent agent is homogeneously dispersed in the wash composition; and
iii) the acyl isethionate and acyl taurate are present at a weight ratio from 1:3 to 3:1 or from 1:2 to 2:1 or from 1:1.5 to 1.5 to 1 or from 1:1.2 to 1 to 1.2:1 or from 1.15:1 to 1:1.15 or from 1:1.

In a second aspect, the present invention is directed to a method of washing a surface, like skin, with the with the wash composition according to the first aspect of the invention.

In a third aspect, the invention is directed to the use of a PPAR agonist to homogeneously suspend pearlescent agent in a wash composition having surfactant and that is substantially free of a polymeric thickener and polymeric suspending agent.

All other aspects of the present invention will more readily become apparent from the description and Examples which follow.

Skin, as used herein, is meant to include skin on the arms (including underarms), face, back, feet, neck, chest, hands, legs, buttocks and scalp. Stable means the composition remains translucent and iridescent with no visual signs of syneresis, precipitation and pearlescent agent separation after being stored at 22° C. for at least two (2) weeks, and preferably, after being stored at 45° C. for one (1) month, and preferably, two (2) months after making. Stable also means that after being stored at 22° C. for two (2) weeks after making, the wash composition consistent with the invention displays no malodor or color change, excellent fragrance characteristics (i.e., no perceptible fragrance change) and a change in viscosity of no more than 15%. Rich foam profile is defined to mean having a foam volume and wash off characteristics consistent with isotropic washes having no pearlescent agent as determined by skilled panelists using water at about 37° C. and wash composition and water at a weight ratio from 1:6 to 1:3 or from 1:5 to 1:4.5 or from 1:4.5 to 1:4.

Isotropic means a composition with micelles arranged without directional preference. Translucent means not opaque, and therefore, permitting some light to pass through with diffusion such that objects in sight cannot be seen clearly. Iridescent means luminous colors seem to change as product is moved and exposed to light. The wash composition of the present invention is isotropic. Soap, as used herein, means salt of a fatty acid. Zwitterionic means being dipolar, a surfactant having charged groups of opposite signs where the positive charge is typically an ammonium moiety and the negative charge is typically a carboxylate moiety as often displayed by amino acids. Amphoteric surfactant means a surfactant that changes from positive or cationic to negative or anionic as the pH goes from acidic to basic.

The wash composition of the present invention is one which has a viscosity of at least 100 mPa-s where viscosity is taken at 25° C. with a Discovery HR-2 Rheometer using sand blasted plates having a 1000-micron gap and a shear rate of 10⁻¹ s⁻¹. The wash composition of this invention is one suitable to be wiped or washed off, and preferably, washed off with water. Such a composition can be a home care cleaning composition (e.g., for tabletops, glass, toilets, dishes, upholstery, or laundry) but is preferably a shampoo, make-up wash, facial wash, hand wash, or personal care and liquid body wash. In an embodiment of the invention, the wash composition can have a viscosity from 500 to 25,000 mPa-s, and most preferably, from 1,000 to 20,000 mPa-s (or 1,200 to 18,000 mPa-s or 1,500 to 17,000 mPa-s or 1,800 to 16,000 mPa-s or 2,000 to 15,000 mPa-s or from 2,200 to 9,000 mPa-s). When used for hand applications, the wash composition of the present invention will preferably have a viscosity that is at least 500 mPa-s and not higher than 6,000 mPa-s.

The wash composition of this invention may, optionally, comprise medicinal or therapeutic agents, but preferably, is a wash which is for cosmetic and non-therapeutic uses to remove dirt, oil, make-up or the like from surfaces, especially the skin and scalp. In a preferred embodiment, the wash composition is a personal wash composition, and therefore, a liquid face or body wash for use while showering or bathing. The wash composition of the present invention may optionally comprise skin benefit ingredients added thereto such as emollients, vitamins and/or derivatives thereof, resorcinols, retinoic acid precursors, colorants, moisturizers, sunscreens, antibacterial agents, ceramides, mixtures thereof or the like. Such skin benefit ingredients (or agents) can be water and/or oil soluble. If used, oil soluble skin benefit agents typically make up to 2% (or up to 1.5%) by weight of the wash composition with the proviso that PPAR agonist makes up no more than 1% by weight of the wash composition. Water-soluble skin benefit agents, when optionally used, may make up to 10% (or up to 5%) by weight of the wash composition. The wash composition will typically have a pH from 5.8 to 8.2, or from 6 to 7.5, or from 6.1 to 7.2, or from 6.2 to 7 or from 6.2 to 6.8. PPAR agonist means compounds that activate receptors identified as peroxisome-proliferator-activated receptors, a lipid that activates the nuclear receptor PPAR, and particularly, alpha. Homogeneously dispersed pearlescent agent means the agent is spread out evenly throughout the wash composition.

As used herein, “substantially free of” as it relates to sulfate-based surfactants (e.g., sodium lauryl sulfate or sodium pareth sulfate) means less than 1.5%, and preferably, less than 1%, and most preferably, less than 0.5%, even more preferably, less than 0.2% (or less than 0.1%) by weight of the wash composition. In an embodiment of the invention, “substantially free of sulfate-based surfactant includes a wash composition with 0.0% (i.e., none) by weight sulfate-based surfactant. Regarding the non-sulfate-based ingredients (i.e., ingredients, like a hydantoin preservative, phthalate, polymeric thickener, polymeric suspending agent), substantially free of means less than 0.6% by weight, and preferably, less than 0.4%, and most preferably, less than 0.3%, (or less than 0.22% or less than 0.1% or less than 0.05%) by weight of the wash composition of the invention. As to the non-sulfate-based ingredients, substantially free of also includes 0.0% (i.e., none) by weight of the ingredient in the wash composition of the invention. As to soap (e.g., sodium stearate or sodium oleate), the wash composition of this invention can have less than 2%, and preferably, less than 1%, and most preferably, less than 0.75% by weight soap, or less than 0.55%, or less than 0.25%, or from 0.001 to 0.24% or 0.0% (no soap) by weight soap based on total weight of the wash composition. With respect to all ingredients that the wash composition of the present invention may be substantially free of, it is within the scope of the invention to include from 0.001 to 0.045% by weight of such ingredient. In yet another preferred embodiment, the wash composition of the present invention comprises less than 35 ppm, and preferably, less than 25 ppm, and most preferably, less than 15 ppm dioxane or less than 2 ppm or less than 1 ppm dioxane or 0.0 ppm dioxane based on total dioxane in the wash composition. In another embodiment, the wash composition of this invention may comprise from 0.00001 to 0.00005% by weight dioxane, like 1,4-dioxane. Polymeric thickener means an ingredient added to build viscosity in an aqueous system, and for example, a carbomer. Polymeric suspending agent means an ingredient added to an aqueous system to suspend components in the system without building or increasing viscosity, and for example, a suspending additive having glycerin, cellulose and cellulose gum made commercially available as Arbalon™ R-50 from C.P. Kelco.

The term comprising is meant to encompass the terms consisting essentially of and consisting of. For the avoidance of doubt, and for illustration, the wash composition of this invention comprising anionic surfactant and pearlescent agent is meant to include a composition consisting essentially of the same and a composition consisting of the same. All ranges defined herein are meant to include all ranges subsumed therein. Except in the operating and comparative examples, or where otherwise explicitly indicated, all numbers in this description indicating amounts or ratios of materials or conditions and/or physical properties of materials and/or use are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

Acyl isethionate suitable for use may be an alkoxylated isethionate such as those described in Ilardi et al., U.S. Pat. No. 5,393,466, entitled “Fatty Acid Esters of Polyalkoxylated isethonic acid. The isethionate surfactants can include the reaction product of fatty acids esterified with isethionic acid and neutralized with a base like sodium or potassium hydroxide. The acyl isethionate surfactant can have the general formula:

$$RC — O ( O ) — C ( X ) H — C ( Y ) H — {( {OCH}_{2} — {CH}_{2} )}_{w} — {SO}_{3}^{-} {M}^{+} , \qquad I$$

where R is a C₅ to C₁₉ saturated or unsaturated alkyl, w is an integer from 0 to 4 (1 to 4 for the alkoxylated option), X and Y are each independently hydrogen or an alkyl group having 1 to 4 carbons and M is a counter ion such as a Na⁺, K⁺, NH₄⁺ or the like.

It is also within the scope of the invention, when w is 0, for either the carbon alpha or beta to the sulfonate group to have one C₁₋₄ alkyl substitution in place of hydrogen, preferably a C₁₋₃ alkyl substitution and most preferably, a methyl group. It is also within the scope of the invention for R to have a degree of unsaturation, and typically, no more than 2, and preferably, no more than 1 double bond.

Illustrative examples of the isethionates suitable for use in the wash composition include sodium capryl isethionate, sodium caproylyl isethionate, sodium capryl methyl isethionate, sodium caproylyl methyl isethionate, sodium cocoyl isethionate, sodium cocoyl methyl isethionate, sodium lauroyl isethionate, sodium lauroyl methyl isethionate, potassium lauroyl isethionate, potassium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, ammonium cocoyl isethionate, ammonium cocoyl methyl isethionate, or a mixture thereof. In an embodiment of the invention, isethionate selected for use comprises sodium lauroyl isethionate, sodium cocoyl isethionate or a mixture thereof. In another embodiment, the isethionate selected consists essentially of or consists of sodium lauroyl isethionate, sodium cocoyl isethionate or a mixture thereof. In an often-desired embodiment the isethionate used is sodium lauroyl isethionate.

Acyl taurates suitable for use include, for example, those commonly known as sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, mixtures thereof or the like. In an embodiment of the invention, the taurate used is sodium methyl lauroyl taurate.

Should the wash composition comprise optional anionic surfactants used in addition to acyl isethionates and taurates, often selected for use are acyl glycinates and salts thereof. Such glycinates include C₈ to C₂₀, and preferably, C₁₀ to C₁₈ and most preferably, C₁₂ to C₁₆ or C₁₂ to C₁₄ acyl glycinates. Illustrative and nonlimiting examples of the glycinates that may be included are sodium lauroyl glycinate, sodium myristoyl glycinate, sodium cocoyl glycinate, potassium lauroyl glycinate, sodium myristoyl glycinate, potassium cocoyl glycinate or a mixture thereof. In an embodiment of the invention, sodium cocoyl glycinate, potassium cocoyl glycinate or a mixture thereof are often preferred when acyl glycinates are used. In another embodiment, sodium or potassium lauroyl glycinate or both may also be used in the wash composition of the invention. In even another embodiment of the invention, the acyl portion of the glycinates that may be selected for use is preferably saturated but suitable to be unsaturated with no more than 2 double bonds, including conjugated double bonds.

Acyl glutamates (and salts thereof) may also optionally be included as additional anionic surfactants in the wash composition of the present invention. Glutamates such as C₈ to C₂₀, and preferably, C₁₀ to C₁₈ and most preferably, C₁₂ to C₁₆ or C₁₂ to C₁₄ acyl glutamates where the acyl portion is preferably saturated are suitable for optional use. As is the case with glycinates, the hydrophobic portion of the glutamates may be unsaturated with no more than 2 double bonds, including conjugated double bonds. Illustrative yet nonlimiting examples of the glutamates that may be used include sodium capryloyl glutamate, sodium lauroyl glutamate, sodium myristoyl glutamate, sodium cocoyl glutamate, sodium stearoyl glutamate, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium olivoyl glutamate, sodium palmitoyl glutamate, sodium undecylenoyl glutamate, disodium cocoyl glutamate or a mixture thereof. In an embodiment of the invention, sodium lauroyl glutamate, sodium cocoyl glutamate, sodium stearoyl glutamate or a mixture thereof is preferred when glutamates are desired for optional use. In another embodiment, if a glutamate is used, sodium myristoyl and/or sodium cocoyl glutamate may often be desired.

Still other anionic surfactants suitable to be selected for optional use in the wash composition with acyl isethionate and taurate include alkyl sulfates, alkyl sulfonates, alpha-olefin sulfonates, alkyl sulfosuccinates, alkyl ether sulfosuccinates, acyl sarcosinates or a mixture thereof (including any salts thereof).

As noted herein, the wash composition is, preferably, substantially free of surfactants that are sulfated (e.g., sulfate comprising surfactants (e.g., sodium lauryl sulfate) and including those that are alkoxylated (e.g., sodium lauryl ether sulfate)). In an embodiment of the invention sulfated surfactants may be used, and preferably, at an amount of less than 1.5% by weight of the wash composition. If optionally included, the alkyl sulfates can include C₈-C₂₀ alkyl sulfates and/or alkoxylated versions such as sodium lauryl ether sulfate, sodium pareth ether sulfate or a mixture thereof where the ethoxylated portion (EO) is typically on average 1-3 EO groups. When selected, sodium lauryl sulfate, sodium lauryl ether sulfate, sodium pareth sulfate, ammonium lauryl sulfate or a mixture thereof are illustrative examples that may be included for use.

As to anionic sulfonates and their salts that may be selected for optional use (in addition to or in lieu of any other optional anionic surfactants described for use with acyl isethionate and taurate), the same can include alkyl sulfonates, alkyl glyceryl ether sulfonates, alpha olefin sulfonates (hydrocarbons being an alkene, CₓH₂ₓ, with a double bond in the alpha position) or a mixture thereof. Typically, the alkyl portion (or alkene portion in the case of alpha olefin sulfonates) is from C₈-C₂₄, and preferably, from C₁₀-C₂₀, and more preferably, from C₁₂ to C₁₈ or from C₁₂ to C₁₆ or from C₁₄ to C₁₆.

Suitable succinates (including their salts) that may optionally be included are those with a C₁₀ to C₂₀ hydrophobic portion. Illustrative examples include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a mixture thereof. For the avoidance of doubt, MIPA and MEA refer to monoisopropanolamine and monoethanolamine, respectively.

The acyl sarcosinates suitable for optional use include those having a C₈-C₂₀ or C₁₀-C₁₈ or C₁₂-C₁₈ acyl group. Illustrative examples of the sarcosinates that may optional be used include sodium lauroyl sarcosinate, sodium cocoyl sarcosinate, sodium stearoyl sarcosinate or a mixture thereof.

In an embodiment of the invention, at least one anionic surfactant that may be optionally selected is sodium lauryl sulfosuccinate, sodium myristoyl sulfosuccinate, sodium cocoyl sulfosuccinate, sodium stearoyl sulfosuccinate, sodium laureth sulfosuccinate, sodium pareth sulfosuccinate, disodium laureth sulfosuccinate, disodium lauryl sulfosuccinate, diethylhexyl sodium sulfosuccinate or a mixture thereof. In one embodiment of the invention, acyl sarcosinate makes up from 0.5 to 10% or from 1 to 8% or from 3 to 6% or 4 to 5% by weight of the total weight of anionic surfactant in the wash composition with sodium cocoyl sarcosinate, sodium lauroyl sarcosinate or a mixture thereof often being preferred when a sarcosinate is optionally used.

In yet another embodiment, optionally used anionic surfactant may include sodium methyl 2-sulfolaurate or disodium 2-sulfolaurate or both

As to the amphoteric surfactant suitable for use in the wash composition of the invention, the same can include betaines like lauryl betaine, lauryldimethyl betaine, coco betaine, cocamidopropyl betaine, laurylamidopropyl betaine, a mixture thereof or the like.

Still other amphoteric surfactants suitable for use include lauryl hydroxysultaine, cocamidopropyl hydroxysultaine or mixtures thereof. Even other amphoteric surfactants that may be used in the wash composition include alkyl amidopropyl hydroxysultaines including palmityl, stearyl and/or oleyl amidopropyl hydroxysultaine, and most preferably, palmityl amidopropyl hydroxysultaine. Other amphoteric surfactants suitable for use include behenyl betaine, capryl/capramidopropyl betaine, stearyl betaine, myristyl hydroxysultaine or a mixture thereof. In an embodiment of the invention, the amphoteric surfactant used comprises cocamidopropyl betaine, lauryl sulfobetaine, cocodimethyl carboxymethyl betaine, laurylhydroxy sulfobetaine, cocoamidopropylhydroxyl sulfobetaine, lauryl hydroxysultaine, a mixture thereof or the like. Additional amphoteric surfactants that may be used include C₁₀₋₁₈ alkylamidopropylamine N-oxides like laurylamidopropylamine N-oxide, C₁₀₋₁₈ alkyldimethylamine N-oxides like lauryldimethylamine N-oxide, C₁₀₋₁₈ alkylbetaines like laurylbetaine, or mixtures thereof.

In still another embodiment, the amphoteric surfactant used comprises lauryl sulfobetaine, lauryl hydroxysultaine or a mixture thereof.

Still other surfactants suitable for use having charge impacted by pH include cocoyl amine oxide, lauramine oxide, myristamine oxide, palmitamine oxide, stearamine oxide, oleamine oxide, cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof. Additional optional examples of the amphoteric surfactants suitable for use include sodium lauroamphoacetate, sodium cocoamphoacetate, sodium lauroamphoacetate, sodium cocoamphoacetate, cocamphodipropionate or a mixture thereof.

In an embodiment of the invention the amphoteric selected can be at least 80%, and preferably, at least 85%, and most preferably, at least 90% (or 90 to 100% or 94 to 100% or 94 to 98% or 100%) by weight amine oxide whereby such amine oxide can have the formula: R^{x}—N⁺(R^{y})₂—O— where R^{x} is a C₈₋₂₀ alkyl, and preferably, a C₁₀₋₁₈ alkyl, and most preferably, a C₁₂₋₁₈ alkyl (or C₁₂₋₁₆ alkyl) and R^{y} is H or a C₁₋₆ alkyl or C₁₄ alkyl or ‘C₁₋₃ alkyl or C₁₋₂ alkyl or —CH₃. In another embodiment of the invention, amine oxide suitable for optional use is cocoyl amine oxide, lauramine oxide, myristamine oxide, palmitamine oxide, stearamine oxide, oleamine oxide or a mixture thereof. In still another embodiment of the invention, at least 85% or at least 90% or 90 to 100% or 100% by weight of the amphoteric surfactant used will be triglyceride derived and not fatty acid derived.

In yet another embodiment, the amphoteric surfactant used is from 92 to 100% or from 93 to 100% or from 93 to 98% or from 95 to 100% by weight cocamidopropyl hydroxysultaine, cocamidopropyl betaine or a mixture thereof based on total weight of amphoteric surfactant in the wash composition. In still another embodiment, the amphoteric surfactant used is all (100%) cocamidopropyl betaine.

In an embodiment of the invention other surfactants suitable for optional use may include imidazolines, sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion and the same may make up from 0.5 to 2% or from 0.6 to 1.5% or from 0.7 to 1% by weight of the wash composition.

Nonionic surfactants may optionally be used in the wash composition of the present invention. When used, nonionic surfactants are typically present at levels as low as 0.12, 0.2, 0.3, 0.8, 1, 1.3 or 1.5% by weight of the wash composition. The nonionics which may optionally be used include the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine.

In an embodiment of the invention, nonionic surfactants optionally used can include fatty acid/alcohol ethoxylates having the following structures a) HOCH₂(CH₂)ₛ(CH₂CH₂O)ᵥ H or b) HOOC(CH₂)_{c}(CH₂CH₂O)_{d} H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{b} H, where i and k are each independently 5 to 15; and b is 5 to 50. In another embodiment of the invention, i and k are each independently 6 to 12; and b is 15 to 35.

The nonionic may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995 or it may be one of the sugar amides described in U.S. Pat. No. 5,009,814 to Kelkenberg, entitled “Use of N-Poly Hydroxyalkyl Fatty Acid Amides as Thickening Agents for Liquid Aqueous Surfactant Systems” issued Apr. 23, 1991.

In an embodiment of the invention and if used, the nonionic surfactant may comprise an alkyl polyglucoside represented as:

R⁴—O—(S)ₙ, where R⁴ is a C₆ to C₂₀ or C₈ to C₁₈ or C₁₀ to C₁₆ linear or branched alkyl and S is a saccharide group of 5 or 6 carbons such as glucose, xylose, lactose, fructose and/or mannose, and preferably, is glucose, and n represents the degree of polymerization and may have a value of from 1 to 12 or from 1 to 10, and most preferably, from 1.2 to about 1.6 or from 1.3 to 1.5.

In yet another embodiment of the invention, the nonionic surfactant when used includes polysorbate (like polysorbate 20), cocamide monoethanolamine (cocamide MEA) or a mixture thereof. In even another embodiment, optional nonionic makes up from 0.25 to 1% or from 0.3 to 0.85% or from 0.35 to 0.55% by weight of the wash composition. In still another embodiment, the nonionic surfactant when used is cocamide MEA.

Illustrative cationic surfactants suitable for optional use include cetyltrimethylammonium bromide, cetyltrimethylammonium chloride, cetylpyridium chloride, behentrimonium chloride, stearalkonium chloride, lauryltrimethylammonium chloride, mixtures thereof or the like. If used, such surfactants will often make up from 0.01 to 3%, or from 0.1 to 2.5% or from 0.5 to 1% by weight of the wash composition. In an embodiment of the invention, no (0.0%) cationic surfactant is used.

Inorganic salt is an optional ingredient to aid in composition consistency, viscidity and water activity adjustment. Typical salts may be used like NaCl, KCl, MgCl₂, CaCl₂), mixtures thereof or the like. If used, the inorganic salt makes up from 0.01 to 4%, and preferably, from 0.1 to 3%, and most preferably, from 0.25 to 2.2% (or from 0.5 to 1.8%, or from 0.5 to 1.5% or from 0.5 to 1.3% or from 0.6 to 1.2%) by weight of the wash composition.

Fatty acid sources for the surfactants described herein may, for example, include vegetable, soy, nut, jojoba, avocado, olive, seed oil (e.g., sunflower, linseed, rapeseed, moringa), fruit (e.g., mango, papaya, red berries, coconut), palm kernel and/or palm oil. In an embodiment of the invention, less than 5%, and preferably, less than 2%, and most preferably, less than 1% (or 0.0%) palm kernel oil is used to make surfactant included in the present invention. In another embodiment of the invention, from 0.001 to 70%, and preferably, from 0.01 to 60%, and most preferably, from 1 to 50% (or 2 to 39%) by weight of total surfactant used in the present invention is derived from palm oil and/or palm kernel oil. In even another embodiment, surfactant in the present invention is less than 5% by weight (or 0.0% by weight) palm kernel oil derived.

In even another embodiment of the invention, less than 95%, and preferably, less than 90%, and most preferably, less than 85% (or less than 80% or from 5 to 22% or from 6 to 20% or from 10 to 15%) by weight of the hydrophobic portion of surfactant used in the present invention is recovered from petroleum, palm oil, palm kernel oil and/or coconut oil. In still another embodiment, from 90 to 100% by weight of the hydrophobic portion of at least one surfactant used in the present invention is not recovered from petroleum, palm oil, palm kernel oil and/or coconut oil.

In a further embodiment of the invention, from 0.0 to 15% or from 0.05 to 12% or from 0.5 to 10% by weight of any of the surfactants used in the present invention may have hydrophobic portion with carbon recovered from purple carbon, and that is, carbon recovered from carbon dioxide or carbon monoxide or methane waste gas (or mixtures thereof) via biotechnology that utilizes microbial gas fermentation. It is also suitable in the invention to utilize carbon sources recovered from algal oils, microbial enhanced oil recovery processes and from plants like the oil recovered from Macauba pulp.

As to the peroxisome proliferator-activated receptor (PPAR) agonist, the wash composition of the present invention will comprise from 0.1 to 1%, and preferably, from 0.1 to 0.8%, and most preferably, from 0.15 to 0.7% or from 0.18 to 0.7% or from 0.18 to 0.5% or from 0.2 to 0.35% by weight of the PPAR agonist where such agonist is at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist in the wash composition. Other PPAR agonists suitable for use with 12-hydroxystearic acid are preferably PPAR-alpha agonists including but not limited to 10-hydroxystearic acid, pertoselinic acid, conjugated linoleic acid, 12-hydroxy-9-cis-octadecanoic acid (ricinoleic acid), palmitoleic acid, stearolic acid or a mixture thereof.

As noted herein, the wash composition comprises from 0.05 to 1%, and preferably, from 0.07 to 0.5%. and most preferably, from 0.08 to 0.4% or from 0.08 to 0.25%, or from 0.08 to 0.2% or from 0.09 to 0.16%, or from 0.09 to 0.15 or from 0.1 to 0.14% by weight of a pearlescent agent based on total weight of the wash composition. The pearlescent agent comprises from 0 to 100%, and preferably, from 15 to 100%, and most preferably, from 40 to 100% or from 50 to 100%, or from 60 to 100% or from 75 to 100% or from 75 to 90% or from 75 to 85% or from 80 to 95% or from 90 to 99% or from 92 to 100% or from 96 to 100% or 100% by weight glycol distearate. In addition to glycol distearate (i.e., ethylene glycol distearate), ethylene glycol mono-stearate, polyethylene glycol distearate, PEG-3 distearate, bismuth oxychloride or a mixture thereof may optionally be used with or in lieu of glycol distearate.

Polyols (i.e., humectants) are suitable for optional use in the wash compositions of the present invention whereby the same are limited only to the extent that they are suitable for use in a consumer product. Illustrative and nonlimiting examples of the optional polyols suitable for use in the present invention include sorbitol, glycerol (glycerine), mannitol, xylitol, maltitol or mixtures thereof. In an embodiment of the invention, the polyol used is at least 50% by weight glycerol, based on total weight of the polyol used. In another embodiment of the invention, the polyol used is all glycerol (100% by weight). Polyol, when used, will typically make up from 0.5 to 10% by weight of the wash composition, and preferably, from 1 to 7% by weight of the wash composition, and most preferably, from 1 to 6% (or from 1 to 5% or 1 to 4% or from 1.5 to 2.5%) by weight of the wash composition.

Water, as noted, typically makes up from 70 to 92%, and preferably, from 72 to 88%, and most preferably, from 75 to 86% or from 78 to 85% by weight of the wash composition.

Adjusters suitable to modify/buffer the pH may optionally be used. Such pH adjusters include triethylamine, NaOH, KOH, H₂SO₄, HCl, C₆H₈O₇ (i.e., citric acid) or mixtures thereof. The pH adjusters are added at amounts to yield the desired final pH (as defined from 5.8 to 8.2). The pH values may be assessed with commercial instrumentation such as a pH meter made commercially available from Thermo Scientific®. Often the wash composition has a pH from 6 to 7.5, or from 6.1 to 7.2, or from 6.2 to 7 or from 6.2 to 6.8. In an embodiment of the invention, adjuster is typically present at from 0.01 to 0.4% or from 0.02 to 0.3% or from 0.05 to 0.22% by weight of the wash composition.

Lathering aids suitable to align surfactant polar groups can optional be included in the wash composition. Such aids include C₆-C₁₄ fatty acids, or alcohols and/or amide derivatives thereof. Caproic, caprylic, capric, lauric and myristic acid, and any alcohol or amide derivatives of the same may be used in any combination. When used, the lathering aid makes up less than 1.5% or from 0.01 to 1.35% or from 0.5 to 1.3% or from 0.6 to 1.3% by weight of the wash composition. In an embodiment of the invention, no aid (0.0% by weight) is used. In even another embodiment, less than 1.15% by weight of the lathering aid is present. In still another embodiment, the lathering aid is lauric acid.

Long chain fatty acids like C₁₆ to C₂₀ fatty acids may optionally be included in the wash compositions of the invention. When used, such fatty acids make up from 0.05 to 0.8% or from 0.06 to 0.7% or from 0.07 to 0.4 or from 0.08 to 0.3% by weight of the wash composition. Preferred acids suitable for use are palmitic acid, stearic acid, or a mixture thereof. In another embodiment both palmitic acid and stearic acid are used at a weight ratio from 1:3 to 3:1 or from 1:2.5 to 2.5:1 or from 1:2.2 to 2.2:1 or from 1:1. In yet another embodiment, at most 50% by weight of the total weight of C₁₆ to C₂₀ fatty acids will saponify, and therefore, the wash composition of the present invention can comprise 0.8% or less by weight soap, or less than 0.4% or less than 0.2% by weight soap or 0.001 to 0.7% by weight soap or 0.0% by weight soap originating from such long chain fatty acids.

In an embodiment of the invention, while the wash composition preferably does not comprise polymeric suspending agent, the wash composition may optionally comprise suspending agent to additionally contribute to, for example, product homogeneity, user skin appearance and sensory experience. The same, if used, typically makes up less than 0.6% by weight, and preferably, less than 0.4%, and most preferably, less than 0.3%, (or less than 0.22% or less than 0.1% or less than 0.05%) by weight of the wash composition of the invention.

Illustrative examples of the suspending agent suitable for optional use include those which are a fermentation derived cellulose like Arbalon R-50® and Arbalon R-50® EV made available from Lubrizol. Such products comprise glycerine, cellulose and cellulose gum with 1.3 to 2.5 or 1.5 to 2.35 or 1.55 to 2.25 times more cellulose than cellulose gum. Glycerine typically makes up from 2.45 to 3.6 or from 2.55 to 3.5 or from 2.75 to 3.3 times more than the total amount of cellulose and cellulose gum. Often, the desired cellulose is one derived from a microbial fermentation process.

In even another embodiment of the invention, while the wash composition preferably does not comprise polymeric thickening agent, the wash composition may comprise thickening agent to additionally contribute to viscosity and sensory attributes of the wash composition. The same, if used, typically makes up less than 0.6% by weight, and preferably, less than 0.4%, and most preferably, less than 0.3%, (or less than 0.22% or less than 0.1% or less than 0.05%) by weight of the wash composition of the invention.

Illustrative examples of the polymeric thickening agent suitable for optional use include hydroxyethyl cellulose, hydroxypropyl methylcellulose, acrylates/steareth-20 methacrylate copolymer, PEG 120 methyl glucose trioleate (and) propanediol, acrylates/beheneth 25 methacrylate copolymer, xanthan gum, carbomer, acrylates/C10-30 alkyl acrylate crosspolymer, guar hydroxypropytrimonium chloride, carrageenan, xanthan gum/locust bean gum, xanthan gum/guar gum, cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum, Versathix™ (PEG-150 Pentaerythrityl Tetrastearate (and) PPG-2 Hydroxyethyl cocamide and water), Aristoflex AVC (ammonium acryloyldimethyltaurate/VP copolymer), Carbopol® Ultrez 10 and 20 (crosslinked polyacrylic acid); alkaline soluble emulsion polymers such as Aculyn 28, Acuyln 22 or Carbopol Aqua SF1; cationic polymer such as modified polysaccharides including cationic guar available from Aventis under the trade name Jaguar C13S, Jaguar C14S, Jaguar C17, or Jaguar C16; cationic modified cellulose such as UCARE Polymer JR 30 or JR 40 from Amerchol, N-Hance 3000, N-Hance 3196, N-Hance GPX 215 or N-Hance GPX 196 from Ashland, Inc., synthetic cationic polymers such as MerQuat 100, MerQuat 280, Merquat 281 and Merquat 550 by Nalco, cationic starches, e.g., StaLok® 100, 200, 300 and 400 made by Galactasol 800 series by Henkel, Inc., Quadrosoft Um-200, Polyquaternium-24, Structure® ZEA (a hydroxypropyl modified corn starch) or Structure® XL, a cross-linked pregelatinized hydroxypropyl starch phosphate or a mixture thereof.

Oil may optionally be included in the wash composition of this invention. If used, oil could make up from 0.01 to 0.5% or from 0.1 to 0.3% or from 0.1 to 2% by weight of the wash composition. Oils that may be included are any of those suitable to contact skin, and preferably, those that are natural and sustainable. Illustrative examples of such oils include silicone oils and/or mineral oil, but those that are naturally sourced and sustainable like, arachis oil, castor oil, coconut oil, corn oil, cotton seed oil, olive oil, rapeseed oil, safflower seed oil, sesame seed oil, soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, mixtures thereof or the like are typically desired. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like. As noted, however, the wash composition of the present invention is preferably substantially free of silicone oil.

Optional water-soluble cosmetic agents suitable for use in the wash composition of the invention are limited only to the extent that they are capable of being used in a consumer product at the desired pH described.

Illustrative examples of the skin benefit agents suitable to include in the wash composition are acids, like amino acids, such as arginine, valine or histidine. Additional water-soluble benefit agents suitable for use include vitamin B₂, niacinamide (vitamin B₃), vitamin B₅ (Panthenol), vitamin B₆, folic acid (vitamin B₉), vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, water soluble extracts of sage, aloe vera, green tea, grapeseed, thyme, chamomile, yarrow, cucumber, liquorice, rosemary extract or mixtures thereof. Water soluble sunscreens like ensulizole (phenylbenzimidizole sulfonic acid), disodium phenyl dibenzimidazole tetra sulfonate or mixtures thereof may also be included. Total amount of optional water-soluble benefit agents (including mixtures) when present in the wash composition may range from 0.001 to 10%, preferably from 0.001 to 8%, and most preferably, from 0.01 to 7% (or from 1 to 6% or from 1 to 3%) by weight of the wash composition.

It is also within the scope of the present invention to optionally include oil soluble cosmetic agents (i.e., non-water-soluble agents defined as less than 1.0 g of such agent (solute) can be dissolved in 100 ml of 25° C. water (solvent) at atmospheric pressure). The only limitation with respect to such oil soluble cosmetic agents are that the same are suitable for use in a consumer product.

Illustrative examples of the types of oil soluble cosmetic agents that may optionally be used in the wash composition of the invention include components like sunscreens and including but not limited to ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2hydroxybenzoate or a mixture thereof.

Other optional oil soluble cosmetic agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble cosmetic agents suitable for use include omega-3 fatty acids, omega-6 fatty acids, climbazole, farnesol, ursolic acid, myristic acid, geranyl geraniol, oleyl betaine, cocoyl hydroxyethyl imidazoline, vitamin E (and its derivatives including alpha-tocopherol acetate), hexanoyl sphingosine, terpineol, thymol, mixtures thereof or the like.

In an embodiment of the invention, the optional oil soluble cosmetic agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred when used.

When optional oil soluble cosmetic agent is used in the wash composition, such cosmetic agent typically makes up to 2% (or to 1.5%), and preferably, from 0.001 to 1.2%, and most preferably, from 0.05 to 0.85% by weight of the wash composition with the proviso that PPAR agonist makes up no more than 1% by weight of the wash composition.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. Suitable traditional preservatives that may be used include those traditionally found in consumer products. Preservatives suitable for preferred use are iodopropynyl butyl carbamate, phenoxyethanol, hydroxyacetophenone, ethylhexylglycerine, imidazolidinyl urea, sodium dehydroacetate, benzyl alcohol, sodium benzoate and mixtures thereof. Other preservatives suitable for use include sodium dehydroacetate, chlorophenesin and decylene glycol. The preservatives should be selected having regard for the use of the composition and possible incompatibilities between the preservatives and other ingredients included. Preservatives are preferably employed in amounts ranging from 0.01% to 2.0% (or 0.1 to 1.5% or 0.1 to 1.2%) by weight of the total weight of the wash composition. Also preferred is a preservative system with hydroxyacetophenone alone or in a mixture with other preservatives.

While suitable for use, but in an embodiment of the invention, the wash composition is substantially free of hydantoins, parabens and isothiazolinones.

In another embodiment, the preservative used is sodium benzoate. In even another embodiment, 1,2-octanediol is used with preservative like sodium benzoate, phenoxyethanol, iodopropynyl butyl carbamate or a mixture of the same where the vicinal diol typically makes up from 0.5 to 1.5 or from 0.6 to 1.3 or from 0.7 to 1.2 times the amount of preservative used. Such diols are common emollients but are suitable to use to boost preservative efficacy. Lipidated glycines are also suitable for use to boost preservative efficacy. If selected for use, such lipidated component may make up from 0.01 to 0.7% or from 0.1 to 0.6% or from 0.2 to 0.5% by weight of the wash composition. Often preferred lipidated components suitable for use are capryloyl glycine, undecylenoyl or a mixture thereof.

In even another embodiment, the wash composition made according to the invention may comprise thymol, terpineol, chloroxylenol, zinc pyrithione, piroctone olamine or a mixture thereof. In still another embodiment, the wash compositions may comprise N-acetyl methionine, N-acetyl cysteine (D, L isomers) or both. When selected for optional use, the same may make up alone or collectively from 0.01 to 3% or from 0.1 to 2% or from 0.2 to 1.5% or from 0.1 to 1% or from 0.1 to 0.6% by weight of the wash composition.

Fragrances, fixatives, chelators (like EDTA, citric acid, salicylic acid, triethanolamine), colorants (like caramel, beet juice, beta-carotene, vegetable juice or mixtures of the same), and exfoliants may optionally be included in the wash composition. Each of these substances may range from about 0.03 to about 5%, preferably between 0.1 and 3% (or from 0.3 to 1.2%) by weight of the total weight of the wash composition. To the extent exfoliants are used, those selected should be of small enough particle size so that they do not impede the performance of any packaging used to dispense the wash composition of the invention. In an embodiment of the invention, less than 0.7% or less than 0.4% or from 0.1 to 0.3% or 0.0% by weight of the composition wash comprises EDTA.

In another embodiment of the invention, the wash composition will be substantially free of a sulfate-based surfactant and comprise at least one of thiamidol, oleyl adapalenate, hyaluronic acid, 10-hydroxystearic acid, 12-hydroxystearic acid, mandelic acid, lactic acid, caffeine, tretinoin ((all-E)-3,7-dimethyl-9-(2,6,6-trimethyl-1 cyclonexen-1-yl)-2,4,6,8-nonatetraenoic acid), epigallocatechin gallate, adapalene (6-[3-(1-adamantyl)-4-methoxyphenyl]naphthalene-2-carboxylic acid), 2-mecaptonicotinoyl glycine, salicylic acid, benzoyl peroxide, N-acetyl-DL-methionine, terpineol, thymol, glycerine, retinol, retinyl palmitate, retinyl propionate, 4-ethyl resorcinol, 4-hexyl resorcinol, or a mixture thereof. In another embodiment and when used, such ingredients make up (alone or collectively) from 0.0001 to 6%, or from 0.001 to 5%, or from 0.01 to 4% or from 0.01 to 3% or from 0.01 to 2% (or 0.1 to 1.5% or 0.1 to 1.25 or 0.1 to 4.3%) by weight of the wash composition.

In still another embodiment of the invention, weight ratio of total weight of PPAR agonist (TA)/total weight of pearlescent agent (PA) is: TA/PA=0.2 to 1.4, or from 0.3 to 1.2, or from 0.4 to 0.8 or from 0.45 to 0.65.

When making the wash composition, the desired ingredients may be mixed with conventional apparatus under moderate shear and atmospheric conditions, with temperature being from 30 to 85° C. whereby shear continues until a homogeneous product is recovered.

The packaging for the wash composition typically is not limited if composition can be dispensed. In an embodiment on the invention, wash composition is sold in a pouch, bottle, jar, tube or canister. The packaging preferably is biodegradable and/or is prepared from recycled materials including post-consumer resins. Paper bottles are also suitable for use as are those that comprise bamboo. Other suitable formats include impregnating the wash composition in nonwovens like those having cotton, polyester or both or by providing the end use products in water soluble sachets such as those essentially consisting of polyvinyl alcohol.

As mentioned, when using the wash composition of the invention, the composition is often used with water at a composition to water weight ratio from 1:6 to 1:3 or from 1:5 to 1:4.5 or from 1:4.5 to 1:4, contacted with a surface, like skin, and rinsed off with water.

The Examples are provided to facilitate an understanding of the invention. The Examples are not intended to limit the scope of the claimed invention.

### Example I

A wash composition consistent with the invention was made with the components listed in the Table. All components were combined by mixing with moderate shear. Mixing temperature was stabilized between 55 to 80° C. and pressure was atmospheric. The resulting body wash was cooled to about 35° C. and pH was adjusted to 6.5. The percents shown in the Table and referred to in this Example are percents by weight. Citric acid or sodium hydroxide was added at the final stage to adjust pH. The resulting wash composition had a viscosity measured of about 6,500 mPa-s when measured at 22° C.

Stability or stable compositions consistent with the invention had an initial viscosity (i.e., viscosity taken within 24 hours of the composition being made) that changed by no more than 15% after being stored for at about 22° C. for two (2) weeks. Compositions made according to the invention and after being stored at such time and temperature, unexpectedly displayed no malodor, color change, syneresis, or precipitate formation, and surprisingly, maintained an evenly (homogeneously) dispersed pearlescent agent as well as strong fragrance notes consistent with product made within 24 hours, all as determined by trained and skilled panelists. Compositions that failed, not made consistent with the invention, at least showed settling of particles whereby pearlescent agent dropped out of solution. The compositions were not suitable for further evaluation or use. Moreover, after washing with composition consistent with the invention, even after two (2) weeks of storage, all panelists concluded that the wash compositions lathered well, rinsed easily and produced a soft and smooth foam sensation after use, and surprisingly with excellent fragrance notes and pearlescent characteristics.

TABLE

| Water | Balance |
|---|---|
| Cocamidopropyl Betaine | 7.0 |
| Glycerine | 2.1 |
| Sodium Methyl Lauroyl Taurate | 1.3 |
| Sodium Lauroyl Isethionate | 1.3 |
| Sodium benzoate | 1.3 |
| Sodium chloride | 1.1 |
| Fragrance | 0.9 |
| Lauric Acid | 1.1 |
| Cocamide MEA | 0.4 |
| 12 Hydroxystearic Acid | 0.2 |
| Stearic Acid | 0.2 |
| Colorant | 0.2 |
| Preservative Booster | 0.3 |
| Sodium hydroxide | 0.1 |
| Glycol Distearate | 0.1 |
| Palmitic Acid | 0.1 |

### Example II

Wash compositions were made similar to the one described in Example 1 except that no PPAR agonist was included with the balance made up with water. After two (2) weeks of making the wash composition and as determined by visual inspection from trained panelists, all concluded the wash compositions not made consistent with the invention (i.e., no PPAR agonist present) had separation, visual particulate and were not suitable for use.

### Example III

Wash compositions were made similar to the one described in Example 1 except that the PPAR agonist (TA) and pearlescent agent (PA) were present at a weight ratio that was outside of the ratio TA/PA=0.2 to 1.4, the acyl isethionate and acyl taurate were not at a weight ratio from 1:3 to 3:1, or both, and any balance was made up with water. After two (2) weeks of making the wash compositions and as determined by visual inspection from trained panelists, all concluded the wash compositions not made consistent with the invention had separation, visual particulate (pearlescent agent) and were not suitable for use.

What is claimed is:

1. A wash composition comprising:
a. from 1.5 to 4.5% by weight of an anionic surfactant comprising at least 80% by weight of acyl isethionate and acyl taurate based on total weight of the anionic surfactant;
b. from 3 to 10%, by weight amphoteric surfactant;
c. from 0.08 to 1% or from 0.15 to 0.25% by weight of a peroxisome proliferator-activated receptor (PPAR) agonist, the PPAR agonist comprising at least 65% by weight 12-hydroxystearic acid based on total weight of the PPAR agonist;
d. from 0.05 to 1% by weight of a pearlescent agent, the pearlescent agent comprising from 0 to 100% by weight glycol distearate; and
e. from 70 to 92% by weight water,
wherein:
i) the composition is substantially free of polymeric thickener and suspending agent;
i) the pearlescent agent is homogeneously dispersed in the wash composition; and
iii) the acyl isethionate and acyl taurate are present at a weight ratio from 1:3 to 3:1 or from 1:2 to 2:1 or from 1:1.5 to 1.5 to 1 or from 1:1.2 to 1 to 1.2:1 or from 1.15:1 to 1:1.15 or from 1:1.

2. The wash composition according to claim 1 wherein the wash composition further comprises from 0.25 to 1% by weight nonionic surfactant.

3. The wash composition according to claim 1 wherein the PPAR agonist and pearlescent agent are present at a weight ratio of where PPAR agonist/pearlescent agent is from 0.2 to 1.4 and the pearlescent agent is from 15 to 100% by weight glycol distearate.

4. The wash composition according to claim 1 wherein the PPAR agonist further comprises 10-hydroxystearic acid, pertoselinic acid, conjugated linoleic acid, 12-hydroxy-9-cis-octadecanoic acid, palmitoleic acid, stearolic acid or a mixture thereof.

5. The wash composition according to claim 1 wherein the pearlescent agent is 92 to 100% by weight glycol distearate.

6. The wash composition according to claim 1 wherein the wash composition is substantially free of a sulfate-based surfactant, paraben, hydantoin, isothiazolinone, phthalate, soap, acrylate-based thickener, dioxane and silicone oil.

7. The wash composition according to claim 1 wherein the wash composition further comprises 1 to 7% by weight glycerine, from 0.01 to 4% by weight salt, PPAR agonist which is 90 to 100% by weight 12-hydroxystearic acid, and pearlescent agent which is 90 to 100% by weight glycol distearate.

8. The wash composition according to claim 1 wherein the wash composition further comprises thiamidol, oleyl adapalenate, hyaluronic acid, 10-hydroxystearic acid, mandelic acid, lactic acid, caffeine, tretinoin, epigallocatechin gallate, adapalene, 2-mecaptonicotinoyl glycine, salicylic acid, benzoyl peroxide, terpineol, thymol, glycerine, retinol, retinyl palmitate, retinyl propionate, 4-ethyl resorcinol, 4-hexyl resorcinol, or a mixture thereof.

9. The wash composition according to claim 1 wherein the composition is isotropic, has a pH from 5.8 to 8.2, a viscosity from 500 to 25,000 mPa-s, and further comprises sodium benzoate and fatty acid.

10. The wash composition according to claim 1 wherein the wash composition comprises less than 1.5% by weight C₆-C₁₄ fatty acid.

11. A method for washing a surface comprising, in no particularly order, the steps of contacting a surface with the wash composition according to claim 1 and water, and rinsing the composition off with water.

12. The method for washing a surface according to claim 11 wherein the surface is skin or hair.
